(19) United States
(12) Patent Application Publication
(10) Pub. No.: US 2018/0184930 A1
(43) Pub. Date: Jul. 5, 2018

(54) RECONSTRUCTING CURRENT DIPOLE SOURCES FROM MAGNETIC FIELD DATA ON ONE PLANE

(71) Applicant: Xuan Zhong Ni, Santa Rosa, CA (US)

(72) Inventor: Xuan Zhong Ni, Santa Rosa, CA (US)

(21) Appl. No.: 15/396,733

(22) Filed: Jan. 2, 2017

(51) Int. Cl. A61B 5/04; G06F 19/00

(52) U.S. Cl. CPC A61B 5/04007; A61B 5/04008; G06F 19/3437; G06F 19/3406

(57) ABSTRACT

The reconstruction of the current dipole sources in a portion of a body, such as a heart, from measured magnetic field data on a same plane near the heart is accomplished here.

In embodiments, reconstruction of current dipole sources is accomplished by calculating the positions of the possible current dipoles in the heart with respect to the measured data plane and by converting a set of non linear equations to a set of linear equations.

### REFERENCES CITED

### U.S. Patent Documents

U.S. Pat. No. 8,406,848 B2 March 2013 Wu et al.

U.S. Pat. No. 8,553,956 B2 August 2013 Wu et al.

This is a non-provisional patent application based on the provisional patent application Ser. No. 62/274,778, filed on Jan. 5, 2016.

### BACKGROUND

Magnetic fields produced by the heart (or brain) is measured on a same plane near heart (or brain) by a magnetocardiography (or magnetoencephalography).

Magnetic field data has been used to shows the distribution of the magnetic field obtained at specific measurement points and precise moments of time. Attempts have been made to reconstruct the current dipole sources of interest. Solving this inverse problem is very complex due to the non linear relationship between the measured data and the current dipole sources.

### SUMMARY OF THE INVENTION

The reconstruction of the current dipole sources from the measured magnetic field data is accomplished here. Aspects of the present invention include a computer program product which implementing a methods for calculating the positions of the possible current dipoles from the given magnetic field data on a plane in order to convert a set of non linear equations to a set of linear equations.

### BRIEF DESCRIPTION OF THE DRAWINGS

FIG. 1 shows a single dipole underneath a plane, x-y plane, where the magnetic fields being measured along the plane.

FIG. 2 shows a single dipole underneath a plane, x-y plane, and the 36 points on the plane where the magnetic fields being measured.

FIG. 3 shows closed loop contours of equal value of the measured magnetic fields data by interpolation and extrapolation on the plane.

FIG. 4 shows extreme value points of the magnetic fields data by the steepest decent method calculated from the contours.

FIG. 5 shows extreme value points and the closed loop contours calculated from the magnetic field data due to two current dipoles presented at the same time.

### DETAILED DESCRIPTION OF THE PREFERRED EMBODIMENTS

Magnetic fields produced by the heart (or brain) is measured on a same plane near heart (or brain) by a magnetocardiography (or magnetoencephalography).

Magnetic field data has been used to shows the distribution of the magnetic field obtained at specific measurement points and precise moments of time. Attempts have been made to reconstruct the current dipole sources of interest. Solving this inverse problem is very complex due to the non linear relationship between the measured data and the current dipole sources.

$${B}_{z} ( {\vec{r}}_{k} ) = \frac{{μ}_{0}}{4 π} {Σ}_{n} \frac{{( {\vec{J}}_{n} ( {\vec{p}}_{n} ) \times ( {\vec{r}}_{k} - {\vec{p}}_{n} ) )}_{z}}{{\vert {\vec{r}}_{k} - {\vec{p}}_{n} \vert}^{3}} \qquad ( 1 )$$

here \vec{r}ₖ are the space points of the measured magnetic field on a same plane and \vec{J}ₙ(\vec{p}ₙ) are the current dipoles at the space point \vec{p}ₙ. Here we assume the measured plane is x-y plane with the vertical coordinate z=0 and the current dipoles are under the plane with negative z coordinates.

For simplify here we replace B_{z}(\vec{r}ₖ) by Bₖ, the vector \vec{r}ₖₙ as (\vec{r}ₖ−\vec{p}ₙ) and its absolute value rₖₙ here |\vec{r}ₖ−\vec{p}ₙ|. here \vec{r}ₖ coordinates as (xₖ, yₖ, zₖ) and \vec{p}ₙ coordinates as (xₙ, yₙ, zₙ). We get the following coordinates of \vec{r}ₖₙ as:

xₖₙ=xₖ−xₙ (2)

yₖₙ=yₖ−yₙ (3)

zₖₙ=zₖ−zₙ=−zₙ (4)

We will rewrite the above equation as following,

$${B}_{k} = \frac{{μ}_{0}}{4 π} {Σ}_{n} \frac{( {J}_{nx} \times {y}_{kn } - {J}_{ny} \times {x}_{kn} )}{{( {x}_{kn}^{2} + {y}_{kn}^{2} + {z}_{kn}^{2} )}^{\frac{3}{2}}} \qquad ( 5 )$$

We find that the magnetic field measured are linearly depended on the magnitudes of the current dipoles and non linearly depended on the space positions of the dipoles. Using least square method to find these current dipoles to best fitting the measured magnetic field data, we define the following function;

$$\begin{matrix}F ( {J}_{nx} , {J}_{ny} , {x}_{n} , {y}_{n} , {z}_{n} ) = {Σ}_{k} ( \frac{{μ}_{0}}{4 π} {{Σ}_{n} ( \frac{( {J}_{nx} \times {y}_{kn } - {J}_{ny} \times {x}_{kn} )}{{( {x}_{kn}^{2} + {y}_{kn}^{2} + {z}_{kn}^{2} )}^{\frac{3}{2}}} - {B}_{k} )}^{2} & ( 6 ) \\ \frac{\partial F}{\partial {J}_{Nx}} = 2 ( {Σ}_{k} \frac{{μ}_{0}}{4 π} {Σ}_{n} \frac{( {J}_{nx} \times {y}_{kn } - {J}_{ny} \times {x}_{kn} )}{{( {x}_{kn}^{2} + {y}_{kn}^{2} + {z}_{kn}^{2} )}^{\frac{3}{2}}} - {B}_{k} ) ( \frac{{y}_{kN}}{{r}_{kN}^{3}} ) & ( 7 ) \\ \frac{\partial F}{\partial {J}_{Ny}} = 2 ( {Σ}_{k} \frac{{μ}_{0}}{4 π} {Σ}_{n} \frac{( {J}_{nx} \times {y}_{kn } - {J}_{ny} \times {x}_{kn} )}{{( {x}_{kn}^{2} + {y}_{kn}^{2} + {z}_{kn}^{2} )}^{\frac{3}{2}}} - {B}_{k} ) ( - \frac{{x}_{kN}}{{r}_{kN}^{3}} ) & ( 8 )\end{matrix}$$

Here we do not take partial derivatives with respect to the coordinates of the current dipoles since later cone will show that they can be calculated directly from the measured magnetic field data. From the least square method the above two partial derivatives should be equal zero and we get the following equations.

$$\begin{matrix}( \frac{{μ}_{0}}{4 π} ) {Σ}_{n} ( {Σ}_{k} \frac{{y}_{kn} {y}_{kN}}{{r}_{kn}^{3} {r}_{kN}^{3}} {J}_{nx} - {Σ}_{k} \frac{{x}_{kn} {y}_{kN}}{{r}_{kn}^{3} {r}_{kN}^{3}} {J}_{ny} ) = {Σ}_{k} {B}_{k} \frac{{y}_{kN}}{{r}_{kN}^{3}} & ( 9 ) \\ ( \frac{{μ}_{0}}{4 π} ) {Σ}_{n} ( {Σ}_{k} \frac{{y}_{kn} {x}_{kN}}{{r}_{kn}^{3} {r}_{kN}^{3}} {J}_{nx} - {Σ}_{k} \frac{{x}_{kn} {x}_{kN}}{{r}_{kn}^{3} {r}_{kN}^{3}} {J}_{ny} ) = {Σ}_{k} {B}_{k} ( \frac{{x}_{kN}}{{r}_{kN}^{3}} ) & ( 10 )\end{matrix}$$

FIG. 1 shows one single dipole 2 involved, it is assumed that the dipole is at space point (0, 0, z₀), (z₀<0 here), below the measured plane, x-y plane 1, and the dipole lied in the x-direction.

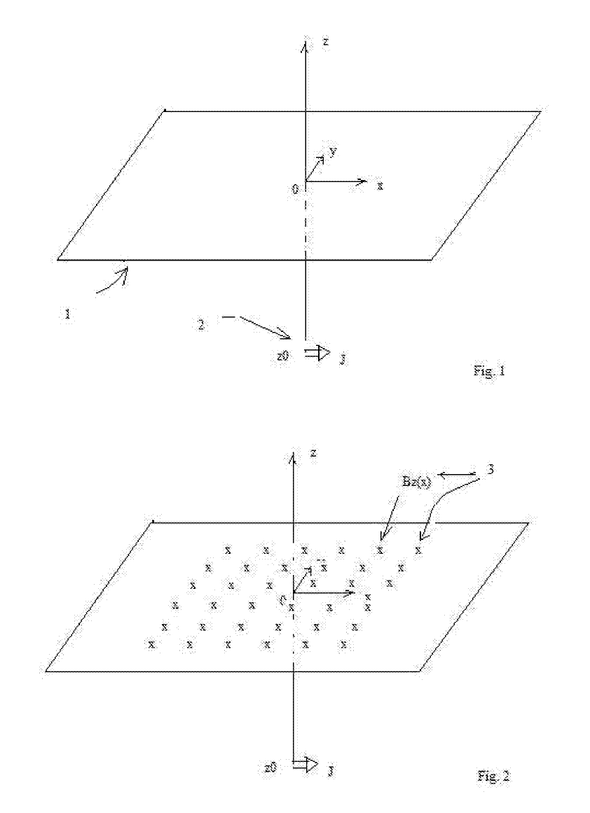

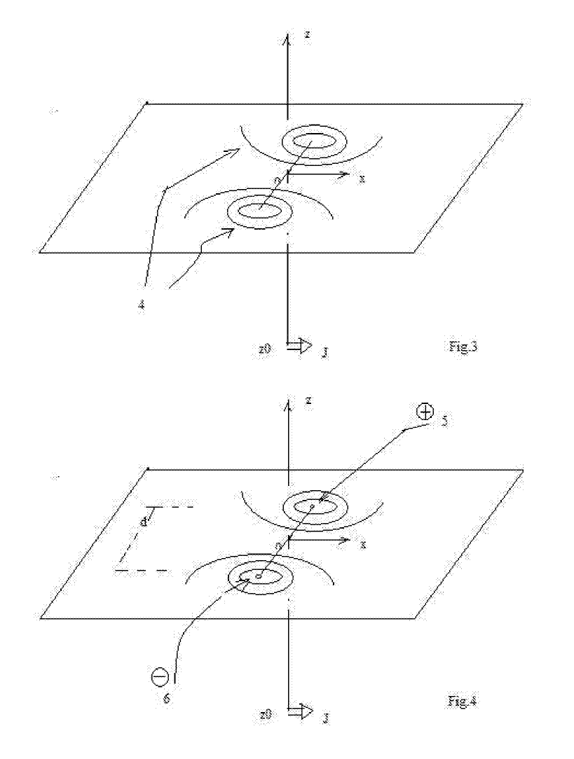

The magnetic fields of z-direction are measured at the 36 (6×6) points 3 on the x-y plane as shown in FIG. 2. Even with such low density points of measurement, it is still very accuracy to construct closed loop contours 4 with equal values of magnetic fields B_{z}(x) by interpolation and extrapolation as shown in FIG. 3.

From the closed loop contours we can determine the extreme value points of B_{z}(x) 5 and 6 in FIG. 4 by the steepest decent calculation, positive sign of extreme point 5 and negative sign of extreme point 6 are shown.

The distance between the two extreme value points, d, can be calculated by the extreme points of B_{z}(x).

Inversely |z₀|=d/√2, and the x, y are the coordinates of the dipole at the mid-point of two extreme points. Also the orientation of the dipole is perpendicular to the connection line and the direction of the dipole is decided by the right hand rule of electric magnetic theory.

It is obvious that the geometric pattern of the closed loop contours is independent to the magnitude of the dipole, |Jₓ|, and is only depended on the value of |z₀| and the orientation of the dipole.

Generally there are more than one current dipole involved, and the present invention include a criterion to determine the number of dipoles involved and their locations.

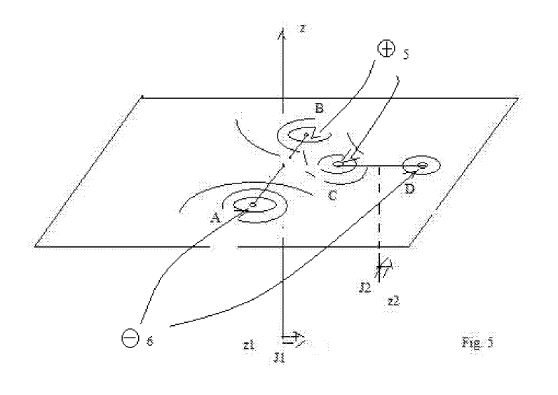

The geometric pattern of one single dipole as in FIG. 3 is symmetric to the x-axis except the positive and negative sign of the magnetic field values. When a second dipole added in the near side of the first one, the pattern become more complicated as in FIG. 5. Adding a second dipole will not change the original locations of the two extreme points by the first dipole, but the values of the two original extreme value points changed. There will be two new extreme value points (one positive extreme maximums by sign of “+” and one negative minimums by sign of “−”) by the second dipole appeared as shown by the new contours from the new measurement data in FIG. 5.

Criterion 1: Find all the extreme value points from the closed loop contours.

Criterion 2: pairing the extreme value points up according to the following principles;

(a) a narrow range of z₀ values can be pre-determined as the heart vertical position below the measured plane, so is a narrow range of d values is determined.

For example from the pattern in FIG. 5, Paring AB and DC are accepted because both lengths are within the narrow range allowed. But pairing AC and DB are not accepted, since if AC is in the range while DB is too long, or if DB is in the range while AC is too short. Pairing two extreme points is only for one positive and one negative. Notes here that points B and C are positive points 5 and points A and D are negative points 6 in FIG. 5.

(b) the current dipoles might appear in different timing frames in the heart activity, the earlier appeared current dipole will maintain its value of z₀ value from its precede temporal frame pattern which distinguishes from the current temporal frame pattern, as comparing FIG. 4 and FIG. 5.

It is assumed that the FIG. 4 is a frame 10 ms ahead of FIG. 5 frame as the first dipole at the location (0, 0, z₁) with x-direction appears 10 ms ahead of the second dipole appearance at a location (x₂, 0, z₂) with y-direction.

It is obvious to pair the extreme points in FIG. 5 by pairing the AB first and then the rest DC is chosen to be a pair.

What is claimed is:

1. A computer program product to execute a method to reconstruct the temporal current dipole sources from the dipoles' magnetic field measured on a same plane; the method comprising:
(a) Closed loop contours of equal values of magnetic fields are calculated by interpolation and extrapolation and plotted on the plane;
(b) Extreme value points are found by the steepest decent method and depicted on the plane;
(c) Pairing the extreme value points according to certain criterions are made and the space locations of the current dipole resources are derived from them;
(d) Converting the non linear equations which are derived from the least square method to the linear equations and solving the linear equations straight forward by the matrix method.

2. The criterions of claim 1 for pairing up the extreme value points according to the allowed narrow range of the lengths connecting the paired extreme value points and the subsequent frame pictures comparison of the dynamic changes of the contours and the number of the extreme value points.

3. A magnetocardiography implementing the computer program product of claim 1.

4. A magnetoencephalography implementing the computer program product of claim 1.
